(19) United States
(12) Patent Application Publication
(10) Pub. No.: US 2003/0087938 A1
(43) Pub. Date: May 8, 2003

(54) Treatment of chronic pain with 3-heterocycloxy-and 3-cycloalkyloxy-3-phenylpropanamines

(76) Inventor: David W. Robertson, Ann Arbor, MI (US)

Correspondence Address: PHARMACIA & UPJOHN, 301 HENRIETTA ST, 0228-32-LAW, KALAMAZOO, MI, 49007, US

(21) Appl. No.: 10/207,511

(22) Filed: Jul. 29, 2002

Related U.S. Application Data
(60) Provisional application No. 60/309,084, filed on Jul. 31, 2001.

(51) Int. Cl. A61K 31/44; A61K 31/426; A61K 31/381; A61K 31/137; A61K 31/34

(52) U.S. Cl. 514/357; 514/651; 514/365; 514/438; 514/471

(57) ABSTRACT

This application relates to the use of certain 3-heterocyclo and 3-cycloalkyloxy-3-phenylpropanamines in the treatment of chronic pain, including neuropathic pain.

### CROSS-REFERENCE TO RELATED APPLICATIONS

This application claims the benefit of the following provisional application(s): U.S. Serial No. 60/309,084, filed Jul. 31, 2001, under 35 USC 119(e)(i).

### BACKGROUND OF THE INVENTION

Chronic painful conditions, in various forms, affect a considerable number of people including, according to the WHO, 4 million cancer sufferers who, worldwide, suffer as a result of a lack of suitable care. There are a number of other conditions, such as musculoskeletal or vertebral pain, neurological pain, headaches or vascular pain. Neurophathic pain, a chronic pain condition occurring in the setting of nervous system injury or tissue injury, is characterized by unusual sensory experiences (allodynia, hyperalgesia) and abnormal pain processing in the central and peripheral nervous systems; treatment of neuropathic pain is difficult. Painful diabetic neuropathy is one of the most frequent complication of diabetes in humans, post-herpetic neuralgia develops in 10-30% of patients after herpes zoster, phantom limb and stump pain is a common sequela of amputation. Chronic pain may also be caused by a trauma, an entrapment neuropathy (e.g. carpal tunnel syndrome), multiple sclerosis or a polyneurophathy associated with AIDS, alcoholism, hypothyroidism, or anticancer chemotherapy.

Conventional treatments of pain fall into two categories: 1) nonsteroidal anti-inflammatory drugs (NSAIDS), used to treat mild pain, but whose therapeutic use is limited by GI adverse effects; and 2) morphine and related opiods, used to treat moderate to severe pain but whose therapeutic use is limited by undesirable side effects including respiratory depression, tolerance, and abuse potential. However, conventional analgesics, whether opiates or NSAIDS's, have limited therapeutic value in the management of chronic pain syndromes. This has led to the use of adjuvant analgesics for the management of these conditions. For example, tricyclic antidepressant are currently the first choice in the treatment of painful diabetic neuropathy. However, few agents are fully effective in all patients and undesirable side effects are common.

### SUMMARY OF THE INVENTION

The present invention provides the treatment of chronic pain with certain 3-heterocyclo and 3-cycloalkyloxy-3-phenylpropanamines. More specifically the present invention relates to the use of compounds of formula I to treat chronic pain

R¹—CH(OAr)—CH₂—CH₂—NR²R³ I

wherein:

Ar is

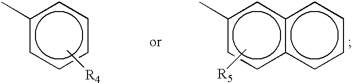

R¹ is C₅-C₇ cycloalkyl, thienyl, halothienyl, (C₁-C₄ alkyl)thienyl, furanyl, pyridyl, or thiazolyl;

each of R² and R³ are independently hydrogen or methyl;

each of R⁴ is independently halo, C₁-C₄ alkyl, C₁-C₃ alkoxy, or trifluoromethyl;

each of R⁵ is independently halo, C₁-C₄ alkyl or trifluoromethyl;

m is 0, 1, or 2;

n is 0 or 1; and

the pharmaceutically acceptable acid addition salts thereof.

The invention also provides for analgesic pharmaceutical formulations for use in the treatment of chronic pain comprising a compound of the above formula and a pharmaceutically acceptable carrier, diluent or excipient therefor.

In the above formula when Ar is phenyl, the substitutent R⁴ group(s) can be attached to the ring at any suitable carbon atom. R thus can represent o-, m- and p-trifluoromethyl; o-, m- and p-fluorophenyl; o-, m- and p-chlorophenyl; o-, m- and p-bromophenyl; o-, m- and p-tolyl; xylyl including all position isomers; o-, m- and p-anisyl; o-, m- and p-tolyl; o-, m- and p-ethoxyphenyl; 2,4-dichlorophenyl; 2,4-difluorophenyl; 2-methoxy-4-chlorophenyl; 2-ethyl-4-bromophenyl; 2,4,6-trimethylphenyl; 2-fluoro-4-trifluoromethylphenyl; 2,4,6-trichlorophenyl; 2,4,5-trichlorophenyl; and the like.

In the above formula when Ar is naphthyl, it can be either 1-naphthyl or 2-naphthyl. The substituent group(s) R⁵ can be attached to the naphthyl ring at any suitable secondary carbon atom. R can thus represent 1-naphthyl; 2-naphthyl; 4-chloro-1-naphthyl; 5-methyl-2-naphthyl; 3-trifluoromethyl-1-naphthyl; 6-iodo-2-naphthyl; 4-methyl-2-naphthyl; 6-n-propyl-1-naphthyl; 2-methyl-1-naphthyl; 6-methyl-1-naphthyl; 4-n-butyl-1-naphthyl; 2-chloro-1-naphthyl; and the like.

The term “halo” means fluoro, chloro, bromo, or iodo.

The term “C₁-C₄ alkyl” means a straight or branched chain alkyl group such as methyl, ethyl, n-propyl, isopropyl, n-butyl, isobutyl, sec-butyl and tert-butyl.

The term “C₁-C₃ alkoxy” means a straight or branched chain alkoxy groups such as methoxy, ethoxy, n-propoxy, and isopropoxy.

The term “C₅-C₇ cycloalkyl” means a cyclic alkyl group containing from 5 to 7 carbon atoms such as cyclopentyl, cyclohexyl and cycloheptyl.

Also included within the scope of this invention are pharmaceutically acceptable salts of the amine bases represented by the above formula formed with non-toxic acids. These acid addition salts include salts derived from inorganic acids such as hydrochloric acid, nitric acid, phosphoric acid, sulfuric acid, hydrobromic acid, hydroiodic acid, nitrous acid, phosphorous acid and the like, as well as salts of non-toxic organic acids including para-toluenesulfonic, methanesulfonic, oxalic, para-bromophenylsulfonic, carbonic, succinic, citric, benzoic and acetic acid, and related inoraganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, choride, bromide, iodine, acetate, propionate, decanoate, caprylate, acrylate, formate, isobutyrate, caprate, heptanoate, priopiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, beta-hydroxybutyrate, glycollate, maleate, tartrate, methanesulfonate, propanesulfonates, naphthalene-1-sulfonate, naphthalene-2-sulfonates, mandelate, and the like salts. Preferred pharmaceutically acceptable acid addition salts include those formed with mineral acids such as hydrochloric acid and hydrobromic acid, and especially those formed with organic acids such as oxalic acid and maleic acid.

The compounds of this invention may be prepared by procedures well known to those of ordinary skill in the art. The preparation of the compounds of the methods of this invention are described in, for example, U.S. Pat. No. 5,023,269.

The carbon atom to which the “R¹” group and “OAr” group is attached is chiral and thus the compounds of the method of this invention exist as stereoisomers. It is within this invention that the single optical isomers are included as well as mixtures of the individual optical isomers including the racemic mixture.

Certain compounds of the methods of this invention are preferred. For example, those compounds wherein Ar is naphthyl, particularly l-naphthyl is preferred. Also preferred are those compounds wherein Ar is phenyl, phenyl substituted with a C₁-C₄ alkyl or C₁-C₃ alkoxy group, particularly unsubstituted phenyl or phenyl substituted by a methyl or methoxy group more particularly unsubstituted phenyl or phenyl substituted at the ortho position with a methyl or methoxy group. Applicant also prefers those compounds of formula I wherein one of R₂ and R³ is hydrogen and the other is a methyl group. Applicant also prefers those compounds of formula I wherein R¹ is thienyl, particularly wherein R¹ is 2-thienyl. Applicant particularly prefers the compounds of formula I wherein Ar is 1-naphthyl, R¹ is 2-thienyl and one of R² and R³ is hydrogen and the other is methyl, that is, the compound known as Duloxetine.

For use in the treatment of chronic pain or neuropathic pain, the compounds of formula I may be administered orally or parenterally in an amount sufficient to alleviate the symptoms of chronic pain, or neuropathic pain. The actual amount of a compound of formula I to be used will vary with the severity and nature of the state of chronic or neuropathic pain, the animal being treated and the level of relief sought. In the human, an oral dose of from about 2 to about 50 milligrams, administered as needed represents appropriate posology. Intramuscular administration of from about 1 to about 25 milligrams provides a dosage comparable to that specified for oral administration.

Pharmaceutical compositions containing a compound of formula I represent an additional aspect of this invention. The active ingredient can be compounded with a pharmaceutically acceptable carrier into any of the usual oral dosage forms including tablets, capsules and liquid preparations such as elixers and suspensions containing various coloring, flavoring, stabilizing and flavor masking substances. For compounding oral dosage forms, the active ingredient can be mixed with various conventional tableting materials such as starches, gum acacia, calcium carbonate, dicalcium phosphate, lactose, dextrose, sucrose, sorbitol, mannitol, alginates, tragacanth, gelatin, calcium silicate, microcrystalline cellulose, methyl cellulose, polyvinylpyrrolidone, water, syrup and talc, magnesium stearate and mineral oil to aid the tableting or capsulating process. Magnesium stearate, as an addition, provides a useful lubricant function when desired.

The active ingredients can be dissolved or suspended in a pharmaceutically acceptable sterile liquid carrier, such as sterile water, sterile organic solvent or a mixture of both. Preferably a liquid carrier is one suitable for parenteral injection. Where the active ingredient is sufficiently soluble it can be dissolved in normal saline as a carrier; if it is too insoluble for this it can often be dissolved in a suitable organic solvent, for instance, aqueous propylene glycol or polyethylene glycol solutions. Aqueous propylene glycol containing from 10 to 75% of the glycol by weight is generally suitable. In other instances other compositions can be made by dispersing the finely-divided active ingredient in aqueous starch or sodium carboxymethyl cellulose solution, or in a suitable oil, for instance arachis oil. Liquid pharmaceutical compositions which are sterile solutions or suspensions can be utilized by intramuscular, intraperitoneal or subcutaneous injection.

Preferably the pharmaceutical composition is in unit dosage form, e.g., as tablets or capsules. In such form, the composition is sub-divided in unit doses containing appropriate quantities of the active ingredient; the unit dosage forms can be packaged compositions, for example, packeted powders or vials or ampoules. The unit dosage form can be a capsule, cachet or tablet itself, or it can be the appropriate number of these in package form. The quantity of the active ingredient in a unit dose of composition may be varied or adjusted from 2 mg or less to 50 mg or more, according to the particular need and the activity of the active ingredient.

As used herein the term “chronic pain” means pain selected from causalgia, neuropathic pain, diabetic neuropathy, post-surgery or traumatic neuropathy, postherpetic neuralgia, peripheral neuopathy, entrapment neuropathy, phantom limb and stump pain, neuropathy caused by alcohol abuse, HIV infection, multiple sclerosis hypothyroidism or anticancer chemotherapy. Applicant particularly prefers the use of the compounds of formula I for the treatment of neuropathic pain.

The term chronic pain relieving amount represents an amount of a compound of formula I which is capable of relieving or reducing chronic pain in a mammal in need thereof.

1. A method of treating chronic pain in a mammal in need thereof which comprises administering to the mammal a chronic pain relieving amount of a compound of the formula
R¹—CH(OAr)—CH₂—CH₂—NR²R³ I
wherein:
Ar is 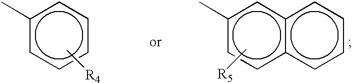
R¹ is C₅-C₇ cycloalkyl, thienyl, halothienyl, (C₁-C₄ alkyl)thienyl, furanyl, pyridyl, or thiazolyl;
each of R₂ and R³ are independently hydrogen or methyl;
each of R⁴ is independently halo, C₁-C₄ alkyl, C₁-C₃ alkoxy, or trifluoromethyl;
each of R⁵ is independently halo, C₁-C₄ alkyl or trifluoromethyl;
m is 0, 1, or 2;
n is 0 or 1; or a pharmaceutically acceptable acid addition salt thereof.

2. The method of claim 1 wherein Ar is napthyl.

3. The method of claim 2 wherein Ar is 1-naphthyl.

4. The method of claim 1 wherein Ar is unsubstituted phenyl, phenyl substituted with a C₁-C₄ alkyl or phenyl substituted with a C₁-C₃ alkoxy.

5. The method of claim 4 wherein Ar is phenyl substituted with a methyl or methoxy.

6. The method of claim 5 wherein the methyl or methoxy group is substituted at the ortho position of the phenyl ring.

7. The method of claim 1 wherein one of R² and R³ is a hydrogen and the other is a methyl.

8. The method of claim 1 wherein Ar is 1-naphthyl, R¹ is 2-thienyl, one of R² and R³ is hydrogen and the other is methyl or a pharmaceutically acceptable acid addition salt thereof.

9. The method of claim 1 wherein Ar is 2-methylphenyl, one of R² and R³ is hydrogen and the other is methyl or a pharmaceutically acceptable acid addition salt thereof.

10. A chronic pain relieving pharmaceutical composition comprising a compound of the formula
R¹—CH(OAr)—CH₂—CH₂—NR²R³ I
wherein:
Ar is 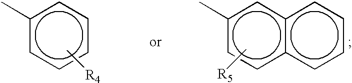
R¹ is C₅-C₇ cycloalkyl, thienyl, halothienyl, (C₁-C₄ alkyl)thienyl, furanyl, pyridyl, or thiazolyl;
each of R² and R³ are independently hydrogen or methyl;
each of R⁴ is independently halo, C₁-C₄ alkyl, C₁-C₃ alkoxy, or trifluoromethyl;
each of R⁵ is independently halo, C₁-C₄ alkyl or trifluoromethyl;
m is 0, 1, or 2;
n is 0 or 1; and
the pharmaceutically acceptable acid addition salts thereof and a pharmaceutically acceptable carrier.

11. A method of treating neuropathic pain in a mammal in need thereof which comprises administering to the mammal a neuropathic pain relieving amount of a compound of the formula
R¹—CH(OAr)—CH₂—CH₂—NR²R³ I
wherein:
Ar is 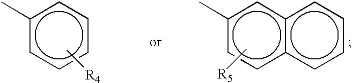
R¹ is C₅-C₇ cycloalkyl, thienyl, halothienyl, (C₁-C₄ alkyl)thienyl, furanyl, pyridyl, or thiazolyl;
each of R² and R³ are independently hydrogen or methyl;
each of R⁴ is independently halo, C₁-C₄ alkyl, C₁-C₃ alkoxy, or trifluoromethyl;
each of R⁵ is independently halo, C₁-C₄ alkyl or trifluoromethyl;
m is 0, 1, or 2;
n is 0 or 1; or a pharmaceutically acceptable acid addition salt thereof.

12. The method of claim 11 wherein Ar is naphthyl.

13. The method of claim 12 wherein Ar is 1-nhaphthyl.

14. The method of claim 11 wherein Ar is unsubstituted phenyl, phenyl substituted with a C₁-C₄ alkyl or phenyl substituted with a C₁-C₃ alkoxy.

15. The method of claim 14 wherein Ar is a phenyl substituted with a methyl or methoxy.

16. The method of claim 15 wherein the methyl or methyl group is substituted at the ortho position of the phenyl ring.

17. The method of claim 11 wherein one of R² and R³ is a hydrogen and the other is a methyl.

18. The method of claim 11 wherein Ar is 1-naphthyl, R¹ is 2-thienyl, one of R² and R³ is hydrogen and the other is methyl or a pharmaceutically acceptable acid addition salt thereof.

19. The method of claim 11 wherein Ar is 2-methylphenyl, one of R² and R³ is hydrogen and the other is methyl or a pharmaceutically acceptable acid addition salt thereof.
